(19) United States
(12) Patent Application Publication
(10) Pub. No.: US 2003/0082120 A1
(43) Pub. Date: May 1, 2003

(54) Method for reducing systemic effects of aging, effects of aging on the skin, and incidence of skin damage from sun exposure using antibiotics of the tetracycline family

(76) Inventor: Harold J. Milstein, Penn Valley, PA (US)

Correspondence Address: Jeffrey Racho, Esq., Harvey & Mortensen, Ste. T-2, 800 Lancaster Avenue, Berwyn, PA, 19312, US

(21) Appl. No.: 10/279,636

(22) Filed: Oct. 24, 2002

Related U.S. Application Data
(60) Provisional application No. 60/348,928, filed on Oct. 26, 2001.

(51) Int. Cl. A61K 31/65; A61K 7/42

(52) U.S. Cl. 424/59; 514/153

(57) ABSTRACT

The present invention is a method of administering an antibiotic of the tetracycline family or a non-antibacterial tetracycline compound in a daily dosage over an extended period of time in order to reduce the effects of aging on a patient's skin, including the appearance of wrinkles; to reduce the systemic effects of aging on the patient, including the incidence of hair loss in men, where such systemic effects appeared to be replaced by a general feeling of vigor, increased energy levels and a more youthful physique; to reduce the effects of sun damage on a patient's skin; to reduce the onset of menopause on female patients; to reduce the incidence of myocardial infarction; to reduce the physiological effects of hypertension; to reduce the incidence stroke, and to reduce the high levels of cholesterol in the patient's bloodstream.

### CROSS REFERENCE TO RELATED APPLICATIONS

This nonprovisional patent application claims the benefit of the filing date of provisional application No. 60/348,928, filed Oct. 26, 2001, which is herein incorporated by reference.

### STATEMENT REGARDING FEDERALLY SPONSORED RESEARCH AND DEVELOPMENT

No Federally sponsored research or development was requested for, accepted, or used in the development related to the invention herein claimed.

### BACKGROUND OF THE INVENTION

1. Description of the Related Art

The tetracycline family of antibiotics comprises broad spectrum antibiotics that are effective against many different bacteria species as well as other species of microorganisms. Tetracyclines exhibit antibacterial effects by inhibiting the ability of the targeted microbe to produce certain proteins. The tetracycline family includes widely prescribed antibiotics such as doxycycline, minocycline, tetracycline, and oxytetracycline.

By the late 1980s, medical researchers had documented substantial therapeutic properties of tetracycline compounds not related to the antibacterial effects of the compound. One example of a therapeutic effect not related to the antibacterial effects of antibiotics of the tetracycline family is evident in the efficacy of such antibiotics in the treatment of the chronic disease periodontitis, a chronic degenerative disease that affects oral connective tissues. Dr. Lorne M. Golub of the State University of New York at Stony Brook documented the non-antibacterial effects of tetracycline in the mediation of the effect of collagenase, an enzyme of the class of enzymes known as matrix metalloproteinases (MMPs), on the gingival tissues of the mouth (see L. M. Golub et al., Minocycline Reduces Collagenolytic Activity During Diabetes: Preliminary Observations and a Proposed New Mechanism of Action, Periodontal Res., 18:516-26 (1983)). Golub's work studied the enzyme-inhibiting properties of antibiotics of the tetracycline family on the tissue-destroying effects of collagenase on the gingival tissues. Collagenase is released into the gingival tissues during the body's inflammatory response to bacterial infection of the gingival tissues and specifically targets the protein collagen. Golub's experiments indicated that antibiotics of the tetracycline family exhibited sufficient enzyme-inhibiting properties to have a therapeutic effect on laboratory rats suffering from periodontitis.

Golub's work also involved non-antibacterial tetracycline compounds known as “chemically modified tetracyclines” (CMTs). CMTs are tetracycline compounds synthesized in such a manner as to negate the antibacterial components of the molecule. CMT molecules include, but are not limited to, 4-dedimethylaminotetracycline (CMT-1), tetracyclinonitrile (CMT-2), 6-demethyl-6-deoxy-4-dedimethylaminotetracycline (CMT-3), 4-dedimethylamino-7-chlorotetracycline (CMT-4), 4-hydroxy-4-dedimethylaminotetracycline (CMT-6), 4-dedimethylamino-12a-deoxytetracycline (CMT-7), 6a-deoxy-5-hydroxy-4-dedimethylaminotet (CMT-8), and 7-dimethylamino-6-demethyl-6-deoxy-4-dedimethylaminotetracycline (CMT-10). Other non-antibacterial tetracycline compounds include 4-dedimethylamino-5-oxytetracycline, 5a,6-anhydro-4-hydroxy-4-dedimethylaminotetracycline, 4-dedimethylamino-11-hydroxy-12a-deoxytetracycline, 12a,4a-anhydro-4-dedimethylaminotetracycline, 6-.alpha.-benzylthiomethylenetetracycline, 7-chlorotetracycline, 5-hydroxytetracycline, 6-demethyl-7chlorotetracycline, 6-demethyl-6-deoxy-5-hydroxy-6-methylenetetracycline, 6-alpha-benzylthiomethylenetetracycline, 6-fluorodemethyltetracycline, 11-alpha-chlorotetracycline, 2-acetyl-8-hydroxy-1-tetracycline and 6-demethyl-6-deoxytetracycline.

CMTs would exhibit the non-antibacterial properties of tetracycline antibiotics, allowing physicians to administer the CMT in lieu of a tetracycline compound to achieve the non-antibacterial efficacious effects of tetracycline antibiotics. This administration of CMTs minimizes the risk of causing undesirable effects related to the antibacterial properties of the tetracycline compound, e.g, contributing to the propagation of antibiotic-resistant strains of bacteria.

Golub and a group of co-inventors were issued U.S. Pat. No. 5,258,371 for a method of treating humans or animals suffering from a condition or disease characterized by excessive collagen destruction comprising the administration of deoxytetracycline (see U.S. Pat. No. 5,238,371, issued Nov. 2, 1993). Golub's basic research has been applied to the pharmaceutical arts by CollaGenex Pharmaceuticals, Inc., a pharmaceutical corporation which developed and markets Periostat™, an FDA-approved drug used in the treatment of periodontitis whose active ingredient is a dosage of doxycycline which is of sufficiently low concentration as to nullify the antibacterial effects of the drug (see CollaGenex Pharmaceuticals, Inc. website <http://www.collagenex.com>).

Other investigators have concluded that antibiotics of the tetracycline family present efficacious effects in the treatment of rheumatoid arthritis (see, e.g., R. Greenwald, Treatment of Destructive Arthritic Disorders with MMP Inhibitors: Potential Role of Tetracyclines, Ann. NY Acad. Sci. 732:181-98 (1994); M. Kloppenburg et al., Minocycline in Active Rheumatoid Arthritis, Arth. And Rheum. 37(5): 629-36 (1994); B. C. Tiley et al., Minoncycline in Rheumatoid Arthitis: A 48-week, Double-blind Clinical Trial, Ann. Intern. Med. 122: 81-89 (1995); see also CollaGenex Pharmaceuticals, Inc. website <http://www.collagenex.com>). Rheumatoid arthritis is a disease characterized by the chronic inflammation of connective tissue in the joints and is also marked by the destruction of collagen by collagenase and gelatinase, another MMP that exhibits collagen-destroying properties.

The work of other researchers indicates that tetracycline compounds may have efficacious effects in the treatment of osteoporosis and cancer. A group of inventors, including Lorne M. Golub, was issued U.S. Pat. No. 4,925,833 in 1990. The '833 patent claimed a method of treating osteoporosis by enhancing the synthesis of bone protein by administering tetracycline compounds (see U.S. Pat. No. 4,925,833, issued May 15, 1990). Dr. Golub was also included in the group of inventors of U.S. Pat. No. 5,837,696, which claimed a method of inhibiting cancer cell growth by the administration of tetracycline, where the cancer is selected from the group consisting of prostate, breast, colon, lung and lymph cancer (see U.S. Pat. No. 5,837,696, issued Nov. 17, 1998).

### SUMMARY OF THE INVENTION

The present invention claims a method of using a dosage of the antibiotic doxycycline or minocycline, or one or more of the antibiotics of the tetracycline antibiotic family, or a non-antibacterial tetracycline compound, including one or more compounds selected from the class of compounds known as chemically modified tetracyclines (CMTs), to: (1) reduce the effects of aging on a patient's skin, including, but not limited to, the appearance of wrinkles and the incidence of keratosis; (2) to further reduce the systemic effects of aging on the patient, including, but not limited to, the incidence of hair loss in men, where such systemic effects appeared to be replaced by a general feeling of vigor, increased energy levels and a more youthful physique; (3) to reduce the effects of sun damage on a patient's skin; (4) to reduce the onset of menopause on female patients; (5) to reduce the incidence of myocardial infarction; (6) to reduce the physiological effects of hypertension; (7) to reduce the incidence stroke, and (8) to reduce the high levels of cholesterol in the patient's bloodstream.

This method comprises the step of administering a daily dosage of tetracycline antibiotics or a non-antibacterial tetracycline compound over an extended period of time to prevent or reduce the onset of the above described symptoms.

### DETAILED DESCRIPTION OF THE INVENTION

Development of Invention

The inventor, a licensed medical doctor specializing in the field of dermatology, has treated numerous patients throughout his medical career. One group of patients consisted of patients from twenty-eight to fifty years of age who suffered from either adult acne (acne vulgaris/adult acne) or rosacea. The inventor usually prescribed an oral dosage of antibiotics of the tetracycline family, primarily doxycycline or minocycline, as a primary treatment of these afflictions.

The inventor's antibiotic treatment consisted of a daily oral dosage of the selected antibiotic for extended periods of time, generally of a term of several years. Traditional antibiotic therapy takes the form of a dosage of antibiotics over a short period of time until the infecting bacteria are killed by the antibacterial effects of the antibiotic compounds. The inventor's administration of the antibiotics in this group of patients, however, consisted of a daily dose over a number of years as the patients suffered from a recurring form of acne which periodically required the administration of such a maintenance dosage over extended periods of time. The inventor believes that the medical arts related to the administration of antibiotics have never taught the administration of antibiotics in such maintenance doses for such extended periods of time, instead teaching that the administration of antibiotics should be of a limited duration to prevent the development of strains of bacteria resistant to antibiotics. The inventor's developed method therefore differs markedly from the traditional administration of antibiotics.

The inventor observed that patients undergoing the described antibiotic treatment often seemed to retain a “youthful” appearance, as evidenced by a decrease in the effects of sun damage on the skin, decreased amounts of wrinkles on the skin, a decrease in the incidence of hair loss in men, and a decrease in the incidence of keratosis of the skin. These patients also seemed less likely to suffer from other systemic effects of aging that would normally afflict a typical patient of similar age, as evidenced by the patients retaining more youthful physiques, appearing to be in better physical condition and exhibiting higher energy levels. The inventor concluded from a qualitative assessment of his patients that the administration of doxycycline or monocycline, or other antibiotics of the tetracycline family, could have “anti-aging” therapeutic effects not related to the antibacterial effects of the drug both on the patient's skin and at a systemic level. The inventor observed that these effects continued in the absence of the presence of the symptoms of acne and believes that the effects would manifest themselves during administration to persons who did not suffer from acne.

Additionally, the inventor observed that female patients undergoing the described antibiotic treatments did not appear to undergo menopause at the age at which the effects of menopause begin to manifest themselves and that the described antibiotic treatments appeared to reduce the onset of hair loss in male patients.

The inventor further observed that patients undergoing the described antibiotic treatments did not appear to suffer from heart attacks (myocardial infarction), hypertension, stroke, or high cholesterol levels despite the patients being at risk for these afflictions as indicated by family histories of the diseases.

The existing related art of the field of the invention indicates that the inventor has a rational basis for concluding that doxycycline or minocycline, or other members of the tetracycline family of antibiotics, or non-antibacterial tetracycline compounds, could have therapeutic effects not related to the antibacterial effects of the drug.

The inventor's observations have been included with this application as Table 1, which details the patients' physical age, diagnosis of the ailment for which the patient was treated, dosage and type of antibiotic prescribed by the inventor. Table 1 includes the inventor's qualitative assessment of the patients' clinical age, which was based upon the inventor's assessment of the patient's overall health and appearance of the patient including the efficacious effects observed throughout the course of the patient's treatment.

TABLE 1

| | Actual | | | | Qualitatively |
|---|---|---|---|---|---|
| Patient | Age | | | Years of | Assessed Age |
| ID. | (years) | Diagnosis | Rx/Dosage | Therapy | (years) |
| CS | 35 | adult acne | Doxycycline - one dose 50 mg daily | 6 | 25 |
| MS | 33 | acne | Doxycycline - one dose 100 mg daily | 4 | 24 |
| HF | 32 | acne | Doxycycline - one dose 100 mg daily | 7 | 26 |
| SM | 46 | adult acne | Doxycycline - one dose 100 mg daily | 10 | 30 |
| RS | 40 | Adult acne | Tetracycline - two doses of 500 mg daily | 20 | 25 |
| KM | 25 | acne | Doxycycline - one dose 50 mg daily | 4 | 19 |
| JK | 39 | rosacea | Doxycycline - one dose 50 mg daily | 6 | 30 |
| KS | 28 | acne | Doxycycline - one dose 100 mg daily | 3 | 21 |
| SE | 30 | acne | Doxycycline - one dose 100 mg daily | 3 | 22 |
| MR | 26 | acne | Minocycline - one dose 50 mg daily | 3 | 21 |
| MW | 60 | rosacea | Tetracycline - one dose 500 mg daily | 6 | 45 |
| KP | 38 | acne | Doxycycline - one dose 100 mg daily | 6 | 27 |
| HM | 51 | acne | Doxycycline - one dose 100 mg daily | 25 | 35 |
| CS | 61 | rosacea | Minocycline - one dose 100 mg daily | 5 | 50 |
| ML | 47 | acne | Doxycycline - one dose 100 mg daily | 4 | 35 |

Description of the Method of the Invention

The method comprises the step of the administration of an effective amount of a compound chosen from the group consisting of tetracycline antibiotics and non-antibacterial tetracycline compounds in a daily dosage over an extended period of time. The inventor believes that the effective amount of the chosen compound includes, but is not limited to, dosages in the amount of 5, 10, 25, 50, 100, 200, 500, and 1000 milligrams (mg) per day of the selected compound administered over an extended period of time. The inventor believes that the extended period of time includes durations of greater than one month, greater than six months, greater than one year, greater than five years, greater than ten years, greater than twenty-five years, and greater than fifty years.

Objects of the Invention

It is an object of the present invention to provide a method of reducing the effects of aging on a patient's skin, including, but not limited to, the appearance of wrinkles and the incidence of keratosis, by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing the systemic effects of aging of a patient, including, but not limited to, the reduction in the incidence of hair loss in men, where such reduction in the systemic effects of aging is accompanied by increased energy levels and a more youthful physique, by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing the onset of menopause in female patients by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing the effects of sun damage on a patient's skin by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing the onset of heart disease by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing hypertension by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing the occurrence of stroke by the administration of doxycycline or minocycline, or one or more of the antibiotics of the tetracycline antibiotic family, or a non-antibacterial tetracycline compound, in a daily dosage over an extended period of time.

It is another object of the invention to provide a method of reducing the level of high cholesterol in a patient by the administration of doxycycline or minocycline, or one or more of the antibiotics of the tetracycline antibiotic family, or a non-antibacterial tetracycline compound, in a daily dosage over an extended period of time.

Further objects of the present invention will become apparent to those of ordinary skill in the art based on the disclosure of the invention herein.

Preferred Embodiment of the Invention

The preferred embodiment of the invention disclosed herein is the administration to a human the antibiotic doxycycline in a dose not more than one thousand (1000) milligrams (mg) on a daily basis for a period not less than one year.

What is claimed is:

1. A method for reducing damage to human skin, comprising the step of administering to a human an effective amount of a drug chosen from the group consisting of tetracycline antibiotics and non-antibacterial tetracycline compounds.

2. The method of claim 1, where said damage to human skin is caused by exposure to the sun and comprises the appearance of wrinkles on said skin.

3. The method of claim 1, where said damage to human skin is caused by the disease keratosis.

4. The method of claim 1, where said damage to human skin is caused by the effects of aging and comprises the appearance of wrinkles on said skin.

5. The method of claim 1, where said tetracycline antibiotic is the antibiotic doxycycline administered in said effective amount of not more than 1000 milligrams per day for a period of time not less than one year.

6. A method of reducing the systemic effects of the aging process in a human being, comprising the step of administering to a human an effective amount of a drug chosen from the group consisting of tetracycline antibiotics and non-antibacterial tetracycline compounds.

7. The method of claim 6, where said systemic effect of the aging process is the onset of menopause in human females.

8. The method of claim 6, where said systemic effect of the aging process is the incidence of hair loss in human males.

9. The method of claim 6, where said systemic effect of the aging process is a decrease in the energy level and a decrease in the general feeling of vigor in a human being.

10. The method of claim 6, where said systemic effect of the aging process is a high level of cholesterol in the bloodstream of a human being.

11. The method of claim 6, where said systemic effect of the aging process is hypertension.

12. The method of claim 6, where said tetracycline antibiotic is the antibiotic doxycycline administered in said effective amount of not more than 1000 milligrams per day for a period of time not less than one year.

13. A method of reducing the likelihood of the incidence of one or more of the afflictions selected from the group consisting of stroke and myocardial infarction in a human being, comprising the step of administering to a human an effective amount of a drug chosen from the group consisting of tetracycline antibiotics and non-antibacterial tetracycline compounds.

14. The method of claim 13, where said tetracycline antibiotic is the antibiotic doxycycline administered in said effective amount of not more than 1000 milligrams per day for a period of time not less than one year.
